(19) United States
(12) Patent Application Publication
(10) Pub. No.: US 2009/0012133 A1
(43) Pub. Date: Jan. 8, 2009

(54) Polymorphic Form of -Acetic Acid

(75) Inventor: Gonzalo Alva, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 12/160,215

(22) PCT Filed: Jan. 10, 2007
(86) PCT No.: PCT/US07/60321

§ 371 Date: Jul. 8, 2008

Related U.S. Application Data
(60) Provisional application No. 60/757,701, filed on Jan. 10, 2006.

(51) Int. Cl. C07D 277/30; A61K 31/426

(52) U.S. Cl. 514/365; 548/204

(57) ABSTRACT

The present invention relates to a novel polymorphic form of the compound {2-methyl-4-[4-methyl-2-(4-trifluoromethylphenyl)thiazol-5-ylmethylthio]phenoxy}-acetic acid, methods of preparing it, pharmaceutical compositions and medicaments containing the same, and use of such polymorphs, compositions and medicaments in the treatment of PPAR mediated diseases or conditions.

### FIELD OF THE INVENTION

The present invention relates to a novel polymorphic form of the compound {2-methyl-4-[4-methyl-2-(4-trifluoromethylphenyl)thiazol-5-ylmethylthio]phenoxy}-acetic acid, methods of preparing it, pharmaceutical compositions and medicaments containing the same, and use of such polymorphs, compositions and medicaments in the treatment of PPAR mediated diseases or conditions.

### BACKGROUND TO THE INVENTION

Peroxisome proliferator activated receptor (hereinafter referred to as PPAR) is a known member of the steroid/retinoid/thyroid hormone receptor family of ligand activated transcription factors and is activated, inter-alia, by high micromolar concentrations of certain peroxisome proliferators. Peroxisome proliferator activated receptor alpha (hereinafter referred to as PPARα), peroxisome proliferator activated receptor gamma (hereinafter referred to as PPARγ) and peroxisome proliferator activated receptor delta (hereinafter referred to as PPARδ) have respectively been identified as subtypes of PPARs.

Certain compounds that activate or otherwise interact with one or more of the PPARs have been implicated in the regulation of triglyveride and cholesterol levels in animal models. See, for example, U.S. Pat. Nos. 5,847,008 (Doebber et al.) and 5,859,051 (Adams et al.) and PCT publications WO 97/28149 (Leibowitz et al.), WO99/04815 (Shimokawa et al.) and WO01/00603 (Glaxo Group Ltd). Oliver et al Proc Natl Acad Sci 98, 5306-5311 (2001) reports the raising of serum triglycerides in the obese rhesus monkey following administration of a PPAR delta agonist.

A particularly preferred PPAR delta agonist is {2-methyl-4-[4-methyl-2-(4-trifluoromethylphenyl)thiazol-5-ylmethylthio]phenoxy}-acetic acid and pharmaceutically acceptable salts, solvates, and hydrolyzable esters thereof (formula (I) below):

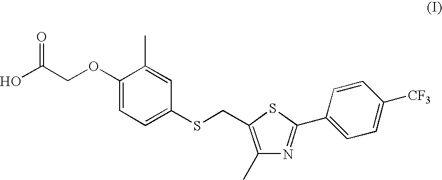

WO01/00603 (the contents of which are incorporated by reference) describes the synthesis of the above compound (hereinafter referred to as the compound of formula (I)). The compound was crystallised from MeOH/water to yield a yellow solid having a melting point of 139-141° C.

Polymorphism is defined as the ability of an element or compound to crystallise in more than one distinct crystalline species. Thus polymorphs are distinct solids sharing the same molecular formula, however since the properties of any solid depends on its structure, different polymorphs may exhibit distinct physical properties such as different solubility profiles, different melting points, different dissolution profiles, different thermal and/or photostability, different shelf life, different suspension properties and different physiological absorption rate. Inclusion of a solvent in the crystalline solid leads to solvates, and in the case of water as a solvent, hydrates.

Polymorphic forms of a compound may be distinguished by x-ray diffraction spectroscopy and other methods including infra-red spectrometry.

### SUMMARY OF THE INVENTION

The present invention provides a polymorph of the compound of formula (I) designated “Form 7”. Form 7 has a melting point of 133±2° C.

As a first aspect, the present invention provides crystalline compound of formula (I) characterized by substantially the same infrared (IR) absorption spectrum as FIG. 1, wherein the IR absorption spectrum is obtained using a Diamond Attenuated Total Reflectance FT-IR spectrometer at 4 cm⁻¹ resolution according to the procedures described herein.

As a second aspect, the present invention provides crystalline compound of formula (I) characterized by an IR absorption spectrum obtained obtained using a Diamond Attenuated Total Reflectance FT-IR spectrometer at 4 cm⁻¹ resolution according to the procedures described herein comprising peaks at five or more positions selected from the group consisting of 2977±2, 2953±2, 2937±2, 1747±2, 1715±2, 1489±2, 1447±2, 1407±2, 1323±2, 1299±2, 1240±2, 1219±2, 1187±2, 1170±2, 1122±2, 1102±2, 1068±2, 1061±2, 1010±2, 894±2, 873±2, 841±2, 811±2 and 747±2 cm⁻¹.

As a third aspect, the present invention provides crystalline compound of formula (I) characterized by an IR absorption spectrum obtained obtained using a Diamond Attenuated Total Reflectance FT-IR spectrometer at 4 cm⁻¹ resolution according to the procedures described herein comprising peaks at 1187±2, 1122±2, 1010±2, 811±2 and 747±2 cm⁻¹

As a fourth aspect, the present invention provides crystalline compound of formula (I) characterized by substantially the same X-ray powder diffraction (XRD) pattern as FIG. 2, wherein the XRD pattern is expressed in terms of 2 theta angles and obtained with a diffractometer using copper Kα-radiation, according to the procedures described herein

As a fifth aspect, the present invention provides crystalline compound of formula (I) characterized by an XRD pattern expressed in terms of 2 theta angles and obtained with a diffractometer copper using Kα-radiation, according to the procedures described herein wherein the XRD pattern comprises 2 theta angles at four or more positions selected from the group consisting of 8.8±0.1, 12.3±0.1, 18.8±0.1, 19.9±0.1, 22.6±0.1, 24.6±0.1, 26.2±0.1, 29.9±0.1, degrees or 10.0, 7.2, 4.7, 4.4, 3.9, 3.6, 3.4, and 3.0 Å d-spacing.

As a sixth aspect, the present invention provides crystalline compound of formula (I) characterized by substantially the same differential scanning calorimetry (DSC) thermograms as FIG. 3 wherein the DSC was performed at a scan rate of 10° C. per minute, using a loosely covered aluminum pan, according to the procedures described herein.

As a seventh aspect, the present invention provides crystalline compound of formula (I) characterized by substantially the same carbon-13 solid-state nuclear magnetic resonance (SSNMR) spectrum for as FIG. 4 wherein the spectrum was acquired at 273K on a spectrometer operating at a proton frequency of 399.87 MHz, a spinning speed of 8 kHz, and a relaxation delay of 10 seconds.

As a eighth aspect, the present invention provides crystalline compound of formula (I) characterized by a carbon-13 solid-state nuclear magnetic resonance (SSNMR) spectrum was acquired at 273K on a spectrometer operating at a proton frequency of 399.87 MHz, a spinning speed of 8 kHz, and a relaxation delay of 10 seconds wherein the SSNMR exhibits resonances at 17.4, 67.5, 125.1, 157.7, and 167.4+/−0.2 ppm.

As a ninth aspect, the present invention provides crystalline compound of formula (I) characterized by substantially the same carbon-13 solid-state nuclear magnetic resonance (SSNMR) spectrum for as FIG. 4 wherein the spectrum was acquired at 273K on a spectrometer operating at a proton frequency of 399.87 MHz, a spinning speed of 8 kHz, and a relaxation delay of 10 seconds wherein the SSNMR exhibits resonances at

17.4, 67.5, 125.1, 157.7, 167.4, 14.3, 32.5, 111.1, 126.7, 128.8, 130.6, 133.0, 135.7, 136.3, 136.8, 152.6, and 170.8+/−0.2 ppm.

As a further aspect, the present invention provides a pharmaceutical composition comprising crystalline compound of formula (I) according to the present invention. The pharmaceutical composition may further comprise one or more pharmaceutically acceptable carriers or diluents.

In a further aspect, the present invention provides a crystalline compound of formula (I) according to the present invention for use in therapy, particularly in the treatment of a disease or condition mediated by one or more human PPAR alpha, gamma or delta (“human PPARs).

In a further aspect, the present invention provides a crystalline compound of formula (I) according to the present invention for use in therapy, particularly in the treatment of dyslipidemia including associated diabetic dyslipidemia and mixed dyslipidemia, syndrome X (as defined in this application this embraces metabolic syndrome), heart failure, hypercholesterolemia, cardiovascular disease including atherosclerosis, arteriosclerosis, and hypertriglyceridemia, type II diabetes mellitus, type I diabetes, insulin resistance, hyperlipidemia, obesity, inflammation, epithelial hyperproliferative diseases including eczema and psoriasis and conditions associated with the lung and gut and regulation of appetite and food intake in subjects suffering from disorders such as obesity, anorexia bulimia, and anorexia nervosa, cancer, Alzheimers disease, multiple sclerosis or other cognitive disorders.

In a further aspect, the present invention discloses a method for prevention or treatment of a disease or condition mediated by one or more human PPARs comprising administration of a crystalline compound of formula (I) according to the present invention.

In a further aspect, the present invention discloses a method for prevention or treatment of dyslipidemia including associated diabetic dyslipidemia and mixed dyslipidemia, syndrome X (as defined in this application this embraces metabolic syndrome), heart failure, hypercholesterolemia, cardiovascular disease including atherosclerosis, arteriosclerosis, and hypertriglyceridemia, type II diabetes mellitus, type I diabetes, insulin resistance, hyperlipidemia, obesity, inflammation, epithelial hyperproliferative diseases including eczema and psoriasis and conditions associated with the lung and gut and regulation of appetite and food intake in subjects suffering from disorders such as obesity, anorexia bulimia, and anorexia nervosa, cancer, Alzheimers disease, multiple sclerosis or other cognitive disorders; comprising administration of a crystalline compound of formula (I) according to the present invention.

In a further aspect, the present invention provides the use of crystalline compound of formula (I) according to the present invention in the preparation of a medicament for the treatment or prophylaxis of a disease or condition mediated by one or more human PPARs.

In a further aspect, the present invention provides the use of crystalline compound of formula (I) according to the present invention in the preparation of a medicament for the treatment or prophylaxis of dyslipidemia including associated diabetic dyslipidemia and mixed dyslipidemia, syndrome X (as defined in this application this embraces metabolic syndrome), heart failure, hypercholesterolemia, cardiovascular disease including atherosclerosis, arteriosclerosis, and hypertriglyceridemia, type II diabetes mellitus, type I diabetes, insulin resistance, hyperlipidemia, obesity, inflammation, epithelial hyperproliferative diseases including eczema and psoriasis and conditions associated with the lung and gut and regulation of appetite and food intake in subjects suffering from disorders such as obesity, anorexia bulimia, and anorexia nervosa, cancer, Alzheimers disease, multiple sclerosis or other cognitive disorders.

### BRIEF DESCRIPTION OF THE DRAWINGS

FIG. 1. The IR spectrum of Form 7 of compound of formula (I) according to the present invention. The x-axis is wavenumber in cm⁻¹ and the y-axis is percent relectance. The IR spectrum is obtained using a Diamond Attenuated Total Reflectance FT-IR spectrometer at 4 cm⁻¹ resolution according to the procedures described herein.

FIG. 2. The XRD pattern of Form 7 of compound of formula (I) according to the present invention. The XRD pattern is expressed in terms of 2 theta angles and obtained with a diffractometer using copper Kα-radiation, according to the procedures described herein.

FIG. 3. The differential scanning calorimetry (DSC) thermogram for Form 7 of compound of formula (I) according to the present invention. DSC was performed at a scan rate of 10° C. per minute, using a loosely covered aluminum pan, according to the procedures described herein.

FIG. 4. The carbon-13 solid-state nuclear magnetic resonance (SSNMR) spectrum for Form 7 of compound of formula (I) according to the present invention. The spectrum was acquired at 273K on a spectrometer operating at a proton frequency of 399.87 MHz, a spinning speed of 8 kHz, and a relaxation delay of 10 seconds.

### DETAILED DESCRIPTION OF THE INVENTION

The present invention provides a novel crystalline form of compound of formula (I) exhibiting one or more advantageous pharmaceutical properties or other advantages over other crystal forms. The crystal form of the present invention possesses is stable at ambient temperatures.

Further desirable properties of the crystalline form of the present invention are the non-hygroscopic nature of this form and its granular crystal habit.

The various forms of compound of formula (I) may be characterized and differentiated using a number of conventional analytical techniques, including but not limited to X-ray powder diffraction (XRD) patterns, infrared (IR) spectra, Raman spectra, differential scanning calorimetry (DSC), thermogravimetric analysis (TGA) and solid state NMR.

“Form 7 of compound of formula (I)” as used herein refers to any of:

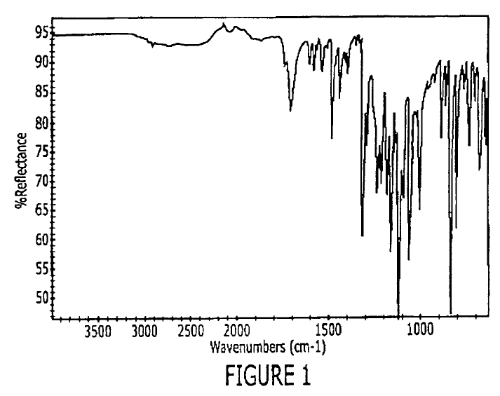

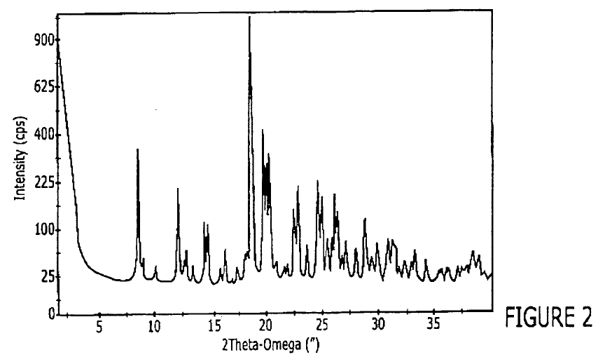

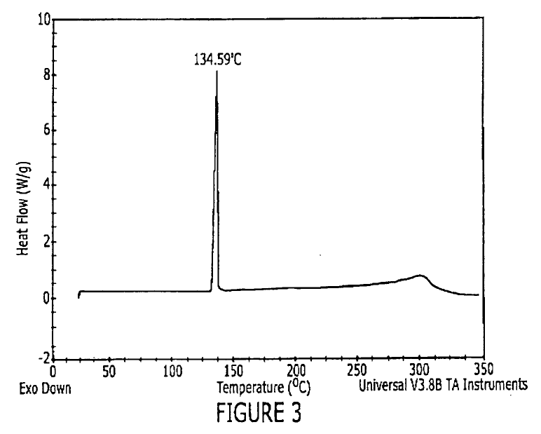

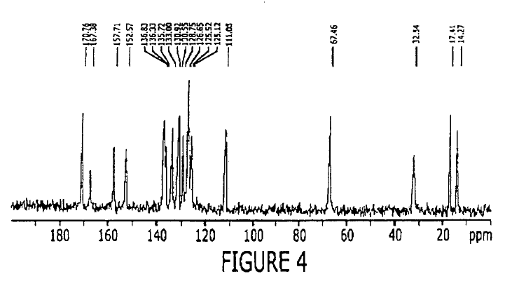

1) a crystalline form of compound of formula (I) characterized by substantially the same infrared (IR) absorption spectrum as FIG. 1, wherein the IR absorption spectrum is obtained using a Diamond Attenuated Total Reflectance FT-IR spectrometer at 4 cm⁻¹ resolution according to the procedures described herein.
2) a crystalline compound of formula (I) characterized by substantially the same X-ray powder diffraction (XRD) pattern as FIG. 2, wherein the XRD pattern is expressed in terms of 2 theta angles and obtained with a diffractometer using copper Kα-radiation, according to the procedures described herein.
3) a crystalline compound of formula (I) characterized by substantially the same differential scanning calorimetry (DSC) thermograms as FIG. 3 wherein the DSC was performed at a scan rate of 10° C. per minute, using a loosely covered aluminum pan, according to the procedures described herein.
4) a crystalline compound of formula (I) characterized by substantially the same carbon-13 solid-state nuclear magnetic resonance (SSNMR) spectrum as FIG. 4 wherein the spectrum was acquired at 273K on a spectrometer operating at a proton frequency of 399.87 MHz, a spinning speed of 8 kHz, and a relaxation delay of 10 seconds

The IR spectrum of the crystalline form of compound of formula (I) according to the present invention (i.e., Form 7) can be determined using conventional equipment and techniques known to those skilled in the art of analytical chemistry and physical characterization. The IR spectra of FIG. 1 was obtained on a Nicolet 550 Magna-IR equipped with a Sens IR Durascope DATR (Diamond Attenuated Total Reflectance) accessory at 4 cm-1 resolution. The wave number in cm⁻¹ (x axis) is plotted against percentage reflectance (y axis). Representative peaks observed in the IR spectrum of Form 7 of compound of formula (I) are as follows: 2977±2, 2953±2, 2937±2, 1747±2, 1715±2, 1489±2, 1447±2, 1407±2, 1323±2, 1299±2, 1240±2, 1219±2, 1187±2, 1170±2, 1122±2, 1102±2, 1068±2, 1061±2, 1010±2, 894±2, 873±2, 841±2, 811±2 and 747±2 cm⁻¹.

As will be apparent to those skilled in the art, not all of these peaks are necessary to conclusively identify an analyzed sample as Form 7 compound of formula (I). Form 7 of compound of formula (I) can be identified by the presence of peaks at 5 or more positions selected form the group consisting of 2977±2, 2953±2, 2937±2, 1747±2, 1715±2, 1489±2, 1447±2, 1407±2, 1323±2, 1299±2, 1240±2, 1219±2, 1187±2, 1170±2, 1122±2, 1102±2, 1068±2, 1061±2, 1010±2, 894±2, 873±2, 841±2, 811±2 and 747±2 cm⁻¹. More particularly, at least peaks at 1187±2, 1122±2, 1010±2, 811±2 and 747±2 cm⁻¹ are present, in one embodiment 2, 3 or 4 further peaks are present and in a further embodiment, all of the foregoing peaks are present.

Slight variations in observed peaks are expected based on the specific spectrometer employed and the analyst's sample preparation technique. Some margin of error is present in each of the peak assignments reported above. The margin of error in the foregoing peak assignments is approximately ±2 cm⁻¹.

Since some margin of error is possible in the peak assignments, a useful method of comparing IR spectra in order to identify the particular form of a sample of compound of formula (I) is to overlay the IR spectrum of the sample over the IR spectrum of each of the known forms. For example, one skilled in the art can overlay an IR spectrum of an unknown form of compound of formula (I), obtained using the methods described herein, over FIG. 1 and, using expertise and knowledge in the art, readily determine whether the IR spectrum of the unknown sample is substantially the same as the IR spectrum of Form 7 of compound of formula (I). If the IR spectrum is substantially the same as FIG. 1, the previously unknown form can be readily and accurately identified as From 7 of compound or formula (I).

The X-ray powder diffraction pattern of Form 7 compound of formula (I) can be determined using conventional techniques and equipment known to those skilled in the art of analytical chemistry and physical characterization. The diffraction pattern of FIG. 2 was obtained using copper Kα radiation on a Philips X'Pert Pro diffractometer equipped with a Philips X'Celerator Real Time Multi Strip (RTMS) detector. The sample was packed into a zero background holder and scanned from 2 to 40 °20 using the following acquisition parameters: 40 mA, 40 kV, 0.017° 20 step, 40 s step time. The sample was spun at 25 rpm during analysis.

A powder sample of Form 7 compound of formula (I) was used to produce the XRD pattern of FIG. 2. 2 Theta angles in degrees α-axis) is plotted against peak intensity in terms of the count rate per seconds (y-axis). The XRD pattern for each crystalline form is unique, exhibiting a unique set of diffraction peaks which can be expressed in 2 theta angles (°), d-spacings (A) and/or relative peak intensities.

2 Theta diffraction angles and corresponding d-spacing values account for positions of various peaks in the XRD pattern. D-spacing values are calculated with observed 2 theta angles and copper Kα1 wavelength using the Bragg equation. Slight variations in observed 2 theta angles and d-spacings are expected based on the specific diffractometer employed and the analyst's sample preparation technique. More variation is expected for the relative peak intensities. Large variations of relative peak intensities may be observed due to preferred orientation resulting from differences in crystal morphology. Identification of the exact crystal form of a compound should be based primarily on observed 2 theta angles or d-spacings with lesser importance place on relative peak intensities. To identify Form 7 compound of formula (I) certain characteristic 2 theta angle peaks occur at 8.8±0.1, 12.3±0.1, 18.8±0.1, 19.9±0.1, 22.6±0.1, 24.6±0.1, 26.2±0.1, 29.9±0.1, degrees or 10.0, 7.2, 4.7, 4.4, 3.9, 3.6, 3.4, and 3.0 Å d-spacing.

Although one skilled in the art can identify Form 7 from these characteristic 2 theta angle peaks, in some circumstances it may be desirable to rely upon additional 2 theta angles or d-spacings for the identification of Form 7 compound of formula (I).

Form 7 compound of formula (I) typically exhibits 2 theta angle peaks in addition to the foregoing peaks. For example, Form 7 compound of formula (I) may exhibit 2 theta angle peaks at essentially the following positions: 10.2±0.1, 12.9±0.1, 14.6±0.1, 14.8±0.1, 16.0±0.1, 16.4±0.1, 20.1±0.1, 20.4±0.1, 22.9±0.1, 25.0±0.1, 25.5±0.1 degrees, or about 8.7, 6.8, 6.1, 6.0, 5.5, 5.4, 4.4, 4.3, 3.9, 3.5, 3.4 Å d-spacing.

In one aspect at least 5, particularly 7 and more particularly all of the above are employed to identify Form 7 compound of formula (I).

Based upon the foregoing characteristic features of the XRPD pattern of Form 7 compound of formula (I), one skilled in the art can readily identify Form 7. It will be appreciated by those skilled in the art that the XRPD pattern of a sample of Form 7 compound of formula (I), obtained using the methods described herein, may exhibit additional peaks.

Some margin of error is present in each of the 2 theta angle assignments and d-spacings reported above. The error in determining d-spacings decreases with increasing diffraction scan angle or decreasing d-spacing. The margin of error in the foregoing 2 theta angles is approximately ±0.1 degrees for each of the foregoing peak assignments.

Since some margin of error is possible in the assignment of 2 theta angles and d-spacings, the preferred method of comparing XRPD patterns in order to identify the particular form of a sample of compound of formula (I) is to overlay the XRPD pattern of the unknown sample over the XRPD pattern of a known form. For example, one skilled in the art can overlay an XRPD pattern of an unknown sample of compound of formula (I), obtained using the method described herein, over FIG. 2 and, using expertise and knowledge in the art, readily determine whether the XRPD pattern of the unknown sample is substantially the same as the XRPD pattern of Form 7 of compound of formula (I). If the XRPD pattern is substantially the same as FIG. 2, the previously unknown form can be readily and accurately identified as Form 7.

Differential Scanning Calorimetry (DSC) was performed on a TA instruments Q1000 Differential Scanning Calorimeter equipped with a refrigerated cooling system.

The DSC thermogram plots the differential rate of heating in watts per second against temperature. The DSC thermogram of Form 7 of compound of formula (I) displays a sharp endotherm at 133° C.±2 which corresponds to the melt. The enthalpy of fusion determined by integrating this peak is 102 J/g±5.

Slight variations in the observed peak is expected based on the specific instrument and pan configuration employed, the analyst's sample preparation technique, and the sample size. Some margin of error is present in the peak assignment reported above. The margin of error is approximately ±2° C. for the peak maximum and ±5 J/g for the heat of fusion.

One skilled in the art can determine whether the DSC thermogram of an unknown sample is substantially the same as the DSC thermogram of Form 7 of the compound of formula (I). If the DSC thermogram is substantially the same as FIG. 3 and the peak position and the calculated heat of fusion are substantially the same as those for Form 7, the previously unknown form can be readily and accurately identified as Form 7.

Solid state nuclear magnetic resonance (SSNMR) is yet another conventional analytical technique for identifying the physical characteristics of Form 7 compound of formula (I). The SSNMR of Form 7 is determined using conventional equipment and techniques known to those skilled in the art of analytical chemistry and physical characterisation.

The solid state NMR spectrum of FIG. 4 was obtained on a Bruker Avance 400 system operating at a proton frequency of 399.87 MHz. A Bruker 4-mm triple-resonance magic-angle spinning (MAS) probe was employed. Approximately 25 mg of the sample was packed into 4-mm outer rotors, sealed with a drive tip, and spun at 8 kHz+/−2 Hz under active control. Cross-polarization from proton to carbon-13 nuclei was used to enhance sensitivity. A 2-ms contact time and a power ramp were used [1]. Spinning sidebands were suppressed using a five-pulse TOSS (total suppression of sidebands) pulse sequence [2]. 1H decoupling was performed at ˜105 kHz using the TPPM decoupling pulse sequence [3]. Spectra were referenced to tetramethylsilane (TMS) using hexamethylbenzene as a secondary external carbon-13 reference [4]. The spectrum shown here is the result of approximately sixteen hundred averaged scans using a 10-second relaxation delay. Chemical shift in ppm α-axis) is plotted against intensity (y-axis).

Form 7 compound of formula (I) is characterized by a solid state carbon-13 NMR spectrum having resonances at 17.4, 67.5, 125.1, 157.7, and 167.4+/−0.2 ppm.

Form 7 compound of formula (I) exhibits resonances in addition to the foregoing peaks. For example, Form 7 compound of formula (I) may exhibit resonances at essentially the following positions: 14.3, 32.5, 111.1, 126.7, 128.8, 130.1, 133.0, 135.7, 136.3, 136.8, 152.6, and 170.8+/−0.2 ppm.

Slight variations in observed chemical shifts are expected based on the specific spectrometer employed and the analyst's sample preparation technique. Some margin of error is present in each of the chemical shifts reported above. The margin of error in the foregoing chemical shifts is approximately ±0.2 ppm.

Since some margin of error is possible in the assignment of chemical shifts, the preferred method of comparing SSNMR spectra in order to identify the particular form of a sample of compound of formula (I) is to overlay the SSNMR spectrum of the unknown sample over the SSNMR spectrum of a known form. One skilled in the art can overlay an NMR spectrum of an unknown sample of compound of formula (I), obtained using the methods described herein, over FIG. 4 and, using expertise and knowledge in the art, readily determine whether the NMR spectrum of the unknown sample is substantially the same as the NMR spectrum of Form 7 compound of formula (I).
[1] G. Metz, X. Wu, S. O, Smith, J. Magn. Reson. A 110 (1994) 219-227.
[2] O. N. Antzutkin, Prog. NMR Spectros. 35 (1999) 203-266.
[3] A. E. Bennett, C. M. Rienstra, M. Auger, K. V. Lakshmi, R. G. Griffin, J. Chem. Phys. 103 (1995) 6951-6958.
[4] W. L. Earl, D. L. Vanderhart, J. Magn. Reson. 48 (1982) 35-54.

Any of the foregoing analytical techniques can be used alone or in combination to identify a particular form of compound of formula (I). In addition, other methods of physical characterization can also be employed to identify the characterize Form 7 compound of formula (I). Examples of suitable techniques which are known to those skilled in the art to be useful for the physical characterization of identification of a crystalline form or solvate include but are not limited to melting point, and thermogravimetric analysis. These techniques may be employed alone or in combination with other techniques to characterize a sample of an unknown form of valaciclovir hydrochloride, and to distinguish Form 7 from other forms of compound of formula (I).

The present invention includes Form 7 compound of formula (I) both in substantially pure form and in admixture with other forms of compound of formula (I). By “substantially pure” is meant that the composition comprises at least 90 percent Form 7 compound of formula (I) as compared to the other forms of compound of formula (I) in the composition, more particularly at least 95 percent Form 7 and in one embodiment, at least 97 percent Form 7 compound of formula (I).

While it is possible that, for use in therapy, Form 7 a compound of formula (I), according to the present invention, (either alone or in admixture with other forms of the compound of formula (I)), may be administered as the raw chemical, it is possible to present the active ingredient as a pharmaceutical composition. Accordingly, the invention further provide a pharmaceutical composition comprising Form 7 compound of the formula (I) and one or more pharmaceutically acceptable carriers, diluents, or excipients. The carrier(s), diluent(s) or excipient(s) must be acceptable in the sense of being compatible with the other ingredients of the composition and not deleterious to the recipient thereof. In accordance with another aspect of the invention there is also provided a process for the preparation of a pharmaceutical composition including admixing Form 7 compound of formula (I), with one or more pharmaceutically acceptable carriers, diluents or excipients.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of active ingredient, depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The active ingredient may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active ingredient may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Dosage forms for nasal or inhaled administration may conveniently be formulated as aerosols, solutions, drops, gels or dry powders.

Pharmaceutical compositions adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered, dose pressurised aerosols, nebulizers or insufflators.

For administration by inhalation the compounds according to the invention are conveniently delivered in the form of spray compositions. Spray compositions may for example be formulated as aqueous solutions or suspensions, for example for nebulisation, or as aerosols delivered from pressurised packs, such as a metered dose inhaler, with the use of a suitable liquefied propellant. Aerosol compositions suitable for inhalation can be either a suspension or a solution and generally contain a compound of the invention and a suitable propellant such as a fluorocarbon or hydrogen-containing chlorofluorocarbon or mixtures thereof, particularly hydrofluoroalkanes, especially 1,1,1,2-tetrafluoroethan, 1,1,1,2,3,3,3-heptafluoro-n-propan or a mixture thereof. The aerosol composition may optionally contain additional formulation excipients well known in the art such as surfactants e.g. oleic acid or lecithin and cosolvents eg. ethanol.

Capsules and cartridges for use in an inhaler or insufflator, of for example gelatine, may be formulated containing a powder mix for inhalation of a compound of the invention and a suitable powder base such as lactose or starch. Alternatively, the compound of the invention may be presented without excipients such as lactose.

Aerosol formulations are preferably arranged so that each metered dose or “puff” of aerosol contains a particular amount of a compound of the invention. Administration may be once daily or several times daily, for example 2, 3 4 or 8 times, giving for example 1, 2 or 3 doses each time. The overall daily dose and the metered dose delivered by capsules and cartridges in an inhaler or insufflator will generally be double those with aerosol formulations.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of composition in question, for example those suitable for oral administration may include flavouring agents.

A therapeutically effective amount of active ingredient will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of active ingredient will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of active ingredient.

The compounds of Formula (I) including Form 7 derivatives thereof, are believed to have utility in as a result of activation of hPPARs.

The present invention thus also provides Form 7 compound of Formula (I) for use in medical therapy, and particularly in the treatment of disorders mediated by human PPARs.

hPPAR mediated diseases or conditions include dyslipidemia including associated diabetic dyslipidemia and mixed dyslipidemia, syndrome X (as defined in this application this embraces metabolic syndrome), heart failure, hypercholesterolemia, cardiovascular disease including atherosclerosis, arteriosclerosis, and hypertriglyceridemia, type II diabetes mellitus, type I diabetes, insulin resistance, hyperlipidemia, obesity, inflammation, epithelial hyperproliferative diseases including eczema and psoriasis and conditions associated with the lung and gut and regulation of appetite and food intake in subjects suffering from disorders such as obesity, anorexia bulimia, and anorexia nervosa, cancer, Alzheimers disease, multiple sclerosis or other cognitive disorders. In particular, the compounds of this invention are useful in the treatment and prevention of diabetes and cardiovascular diseases and conditions including atherosclerosis, arteriosclerosis, hypertriglyceridemia, and mixed dyslipidaemia.

A further aspect of the invention provides a method of treatment of a mammal suffering from a disorder mediated by hPPAR, which includes administering to said subject Form 7 compound of Formula (I).

A further aspect of the present invention provides the use of Form 7 of compound of Formula (I) in the preparation of a medicament for the treatment of a disorder mediated by hPPAR.

Form 7 compound of formula (I) for use in the instant invention may be used in combination with other therapeutic agents for example, statins and/or other lipid lowering drugs for example MTP inhibitors and LDLR upregulators. The compounds of the invention may also be used in combination with antidiabetic agents, e.g. metformin, sulfonylureas and/or PPAR gamma, PPAR alpha or PPAR alpha/gamma agonists (for example thiazolidinediones such as e.g. pioglitazone and rosiglitazone). The compounds may also be used in combination with antihypertensive agents such as angiotensin antagonists e.g. telmisartan, calcium channel antagonists e.g. lacidipine and ACE inhibitors e.g. enalapril. The invention thus provides in a further aspect the use of a combination comprising Form 7 compound of formula (I) with a further therapeutic agent in the treatment of a hPPAR mediated disease.

When Form 7 compound of formula (I) is used in combination with other therapeutic agents, the compounds may be administered either sequentially or simultaneously by any convenient route.

The combinations referred to above may conveniently be presented for use in the form of a pharmaceutical composition and thus pharmaceutical compositions comprising a combination as defined above optimally together with a pharmaceutically acceptable carrier or excipient comprise a further aspect of the invention. The individual components of such combinations may be administered either sequentially or simultaneously in separate or combined pharmaceutical compositions.

When combined in the same composition it will be appreciated that the two compounds must be stable and compatible with each other and the other components of the composition and may be formulated for administration. When formulated separately they may be provided in any convenient composition, conveniently in such a manner as are known for such compounds in the art.

When Form 7 compound of formula (I) is used in combination with a second therapeutic agent active against the same hPPAR mediated disease, the dose of each compound may differ from that when the compound is used alone. Appropriate doses will be readily appreciated by those skilled in the art.

The following Examples are intended for illustration only and are not intended to limit the scope of the invention in any way.

The compound of formula (I) may be prepared by the methods described in WO 01/00603 or by the following route:

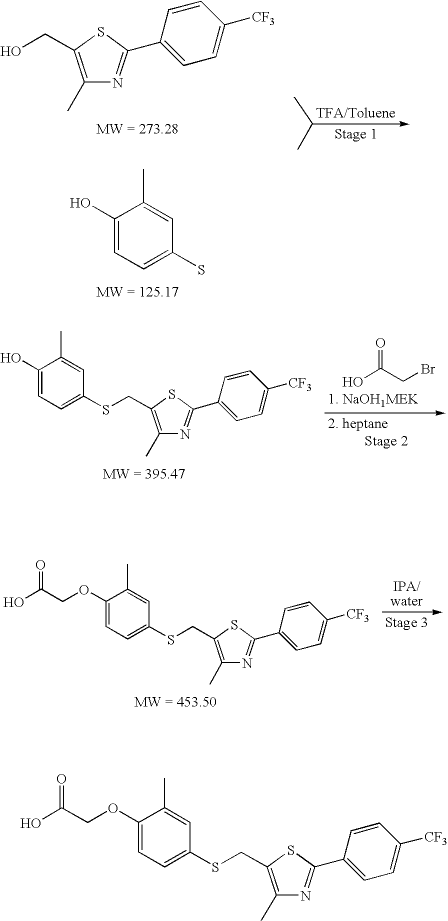

Stage 3 (isolation of Form 7) can be prepared by evaporation from aqueous isopropanol. In particular this is a laboratory scale procedure using a slow cooling crystallisation in aqueous isopropanol.

In a process which is suitable for scaling up, a reactor vessel is charged under N₂ with compound of formula (I) (1.0 wt. 1 eq), isopropanol (4.75 vol) and water (3 vol). The mixture is warmed to ˜70° C. and held for ˜5 min to dissolve solids. The solution is then transferred to a second reactor vessel (pre-heated to 55° C.) via an in-line filter cartridge (30 μm) using isolated vacuum. The first reactor and filtration lines are rinsed with isopropanol (0.25 vol) into the receiving vessel. The filtered solution is re-warmed to ˜70° C. and held for ˜5 min until complete dissolution is observed. The solution is then cooled to ˜57° C. at a rate of about −0.5° C./min. Form 7 seeds (Morphic Form 7, 0.005 wt) are then added via slurry in IPA. The batch is then held at 57° C. for ˜50 minutes. The batch is then cooled to ˜50° C. at a rate of about −0.1° C./min and then to 40° C. at a rate of −0.2° C./min. The batch is then cooled to ˜10° C. at a rate of −0.5° C./min. The batch temperature is maintained at ˜10° C. for at least 30 minutes. The product is collected by filtration under filtered N₂. The cake is washed with cold, pre-filtered IPA/water, 5:3 (2 vol). The product is dried in vaccuo at −55° C. overnight or to constant weight. Expected yield: 90-95%.

### Infrared (IR) Spectroscopy

IR analysis was performed on a Nicolet 550 Magna-IR equipped with a SensIR Durascope DATR (Diamond Attenuated Total Reflectance) accessory. Approximately 2 mg of sample was placed on the diamond probe and flattened using a microscope slide. Pressure was applied to the top of the microscope slide (using the pressure applicator on the Durascope) to ensure the sample underneath had good contact with the probe. Sixty-four co-added scans were collected at 4 cm-1 resolution. A background was collected with no sample on the accessory.

Representative peaks observed in the IR spectrum of Form 7 of compound of formula (I) obtained by DATR were as follows: 2977, 2953, 2937, 1747, 1715, 1489, 1447, 1407, 1323, 1299, 1240, 1219, 1187, 1170, 1122, 1102, 1068, 1061, 1010, 894, 873, 841, 811, 747 cm⁻¹.

The margin of error in the foregoing peak assignments is approximately ±2 cm⁻¹.

### X-Ray Powder Diffraction (XRPD)

The diffraction pattern of FIG. 2 was obtained using copper Kα radiation on a Philips X'Pert Pro diffractometer equipped with a Philips X'Celerator Real Time Multi Strip (RTMS) detector. The sample was packed into a zero background holder and scanned from 2 to 40 °2θ using the following acquisition parameters: 40 mA, 40 kV, 0.017 °2θ step, 40 s step time. The sample was spun at 25 rpm during analysis.

A powder sample of Form 7 of compound of formula (I) was used to produce the XRD pattern of FIG. 2.

Form 7 of compound of formula (I) can be identified by certain characteristic 2 theta angle peaks at 8.8, 12.3, 18.8, 19.9, 22.6, 24.6, 26.2, 29.9 degrees, or 10.0, 7.2, 4.7, 4.4, 3.9, 3.6, 3.4, and 3.0 Å d-spacing.

Further 2 theta angle peaks are at essentially the following positions: 10.2, 12.9, 14.6, 14.8, 16.0, 16.4, 20.1, 20.4, 22.9, 25.0, 25.5 degrees, or about 8.7, 6.8, 6.1, 6.0, 5.5, 5.4, 4.4, 4.3, 3.9, 3.5, 3.4 Å d-spacing.

The margin of error in the foregoing 2 theta angles is approximately ±0.1 degrees for each of the foregoing peak assignments.

### Differential Scanning Calorimetry (DSC)

DSC was performed on a TA instruments Q1000 Differential Scanning Calorimeter equipped with a refrigerated cooling system. The sample was heated in a loosely covered aluminum pan from 25 to 350° C. using a heating rate of 10° C./min.

The DSC thermogram of Form 7 of compound of formula (I) displays a sharp endotherm at 133° C. which corresponds to the melt. The enthalpy of fusion determined by integrating this peak was 102 J/g.

The margin of error is approximately ±2° C. for the peak maximum and ±5 J/g for the heat of fusion.

### Solid State Nuclear Magnetic Resonance (SSNMR)

The solid state NMR spectrum of FIG. 4 was obtained at 273 K on a Bruker Avance 400 spectrometer operating at a proton frequency of 399.87 MHz using a spinning speed of 8 kHz+/−2 Hz and a relaxation delay of 10 seconds.

Form 7 of compound of formula (I) is characterized by a solid state carbon-13 NMR spectrum having resonances at 17.4, 67.5, 125.1, 157.7, and 167.4+/−0.2 ppm.

Form 7 compound of formula (I) exhibits resonances in addition to the foregoing peaks. For example, Form 7 compound of formula (I) may exhibit resonances at essentially the following positions: 14.3, 32.5, 111.1, 126.7, 128.8, 130.6, 133.0, 135.7, 136.3, 136.8, 152.6, and 170.8+/−0.2 ppm.

The margin of error in the foregoing peak assignments is approximately ±0.2 ppm.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A crystalline compound of formula (I) 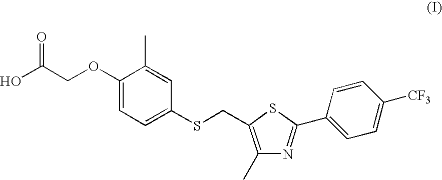
characterized by substantially the same infrared (IR) absorption spectrum as FIG. 1, wherein the IR absorption spectrum is obtained using a Diamond Attenuated Total Reflectance FT-IR spectrometer at 4 cm⁻¹ resolution.

2. A crystalline compound of formula (I) 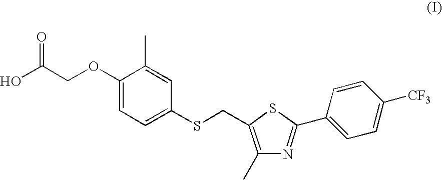
characterized by an IR absorption spectrum obtained using a Diamond Attenuated Total Reflectance FT-IR spectrometer at 4 cm⁻¹ resolution according to the procedures described herein comprising peaks at five or more positions selected from the group consisting of 2977±2, 2953±2, 2937±2, 1747±2, 1715±2, 1489±2, 1447±2, 1407±2, 1323±2, 1299±2, 1240±2, 1219±2, 1187±2, 1170±2, 1122±2, 1102±2, 1068±2, 1061±2, 1010±2, 894±2, 873±2, 841±2, 811±2 and 747±2 cm⁻¹.

3. A crystalline compound of formula (I) 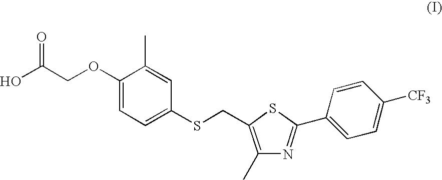
characterized by an IR absorption spectrum obtained using a Diamond Attenuated Total Reflectance FT-IR spectrometer at 4 cm⁻¹ resolution according to the procedures described herein comprising peaks at 1187±2, 11222, 1010±2, 811*2 and 747±2 cm⁻¹.

4. A crystalline compound of formula (I) 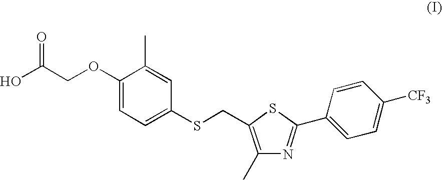
characterized by substantially the same X-ray powder diffraction (XRD) pattern as FIG. 2, wherein the XRD pattern is expressed in terms of 2 theta angles and obtained with a diffractometer using copper Kα-radiation.

5. A crystalline compound of formula (I) 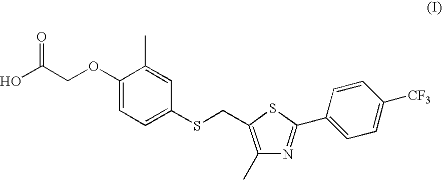
characterized by an XRD pattern expressed in terms of 2 theta angles and obtained with a diffractometer copper using Kα-radiation, according to the procedures described herein wherein the XRD pattern comprises 2 theta angles at four or more positions selected from the group consisting of 8.8±0.1, 12.3±0.1, 18.8±0.1, 19.9±0.1, 22.6±0.1, 24.6±0.1, 26.2±0.1, 29.9±0.1, degrees or 10.0, 7.2, 4.7, 4.4, 3.9, 3.6, 3.4, and 3.0 Å d-spacing.

6. A crystalline compound of formula (I) 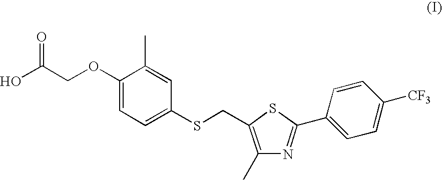
characterized by substantially the same differential scanning calorimetry (DSC) thermograms as FIG. 3 wherein the DSC was performed at a scan rate of 10° C. per minute, using a loosely covered aluminum pan.

7. A crystalline compound of formula (I) 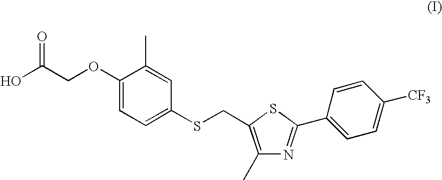
characterized by substantially the same carbon-13 solid-state nuclear magnetic resonance (SSNMR) spectrum for as FIG. 4 wherein the spectrum was acquired at 273K on a spectrometer operating at a proton frequency of 399.87 MHz, a spinning speed of 8 kHz, and a relaxation delay of 10 seconds.

8. A crystalline compound of formula (I) 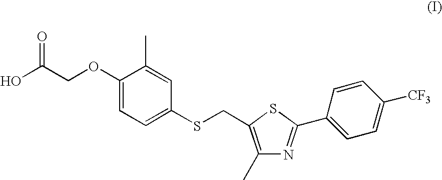
characterized by a carbon-13 solid-state nuclear magnetic resonance (SSNMR) spectrum was acquired at 273K on a spectrometer operating at a proton frequency of 399.87 MHz, a spinning speed of 8 kHz, and a relaxation delay of 10 seconds wherein the SSNMR exhibits resonances at 17.4, 67.5, 125.1, 157.7, and 167.4+/−0.2 ppm.

9. A crystalline compound of formula (I) 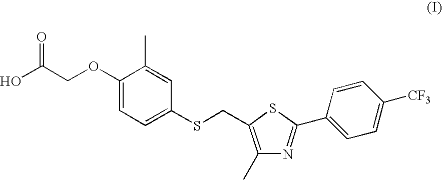
characterized by substantially the same carbon-13 solid-state nuclear magnetic resonance (SSNMR) spectrum for as FIG. 4 wherein the spectrum was acquired at 273K on a spectrometer operating at a proton frequency of 399.87 MHz, a spinning speed of 8 kHz, and a relaxation delay of 10 seconds wherein the SSNMR exhibits resonances at 17.4, 67.5, 125.1, 157.7, 167.4, 14.3, 32.5, 111.1, 126.7, 128.8, 130.6, 133.0, 135.7, 136.3, 136.8, 152.6, and 170.8+/−0.2 ppm.

10-13. (canceled)

14. A pharmaceutical composition comprising a compound according to claim 1.

15. A pharmaceutical composition comprising a compound according to claim 2.

16. A pharmaceutical composition comprising a compound according to claim 3.

17. A pharmaceutical composition comprising a compound according to claim 4.

18. A pharmaceutical composition comprising a compound according to claim 5.

19. A pharmaceutical composition comprising a compound according to claim 6.

20. A pharmaceutical composition comprising a compound according to claim 7.

21. A pharmaceutical composition comprising a compound according to claim 8.

22. A pharmaceutical composition comprising a compound according to claim 9.
